SEQUENCE LISTING (12 sequences)

SEQ ID NO: 1 - DNA, 25 bp, Artificial Sequence
  Synthetic Primer
cctcttacctcagttacaatttata

SEQ ID NO: 2 - DNA, 40 bp, Artificial Sequence
  Synthetic Primer
ggaagtgactaaaccctcacctcattataaattgtaactg

SEQ ID NO: 3 - DNA, 40 bp, Artificial Sequence
  Synthetic Primer
atgaggtgagggtttagtcacttcctataaattgtaactg

SEQ ID NO: 4 - DNA, 40 bp, Artificial Sequence
  Synthetic Primer
tgtacataaccttcgggcatggcactataaattgtaactg

SEQ ID NO: 5 - DNA, 40 bp, Artificial Sequence
  Synthetic Primer
gtgccatgcccgaaggttatgtacatataaattgtaactg

SEQ ID NO: 6 - DNA, 20 bp, Artificial Sequence
  Synthetic Primer
agtgctgcatggacaaagtg

SEQ ID NO: 7 - DNA, 20 bp, Artificial Sequence
  Synthetic Primer
ggttctccacctgttccaga

SEQ ID NO: 8 - DNA, 15 bp, Artificial Sequence
  Synthetic Primer
tataaattgtaactg

SEQ ID NO: 9 - DNA, 30 bp, Artificial Sequence
  Synthetic Primer
gtaatacgactgagtatagggacatttctc

SEQ ID NO: 10 - DNA, 30 bp, Artificial Sequence
  Synthetic Primer
cagttacaatttatagatccgcaccgactc

SEQ ID NO: 11 - DNA, 30 bp, Artificial Sequence
  Synthetic Primer
atttaggtgacactatagaatacaagctac

SEQ ID NO: 12 - DNA, 30 bp, Artificial Sequence
  Synthetic Primer
cagttacaatttatagtttattgcagctta

(19) United States
(12) Patent Application Publication
(10) Pub. No.: US 2019/0000992 A1
(43) Pub. Date: Jan. 3, 2019

(54) Piggyback Delivery of CRISPR/CAS9 RNA into Zebrafish Blood Cells

(71) Applicant: University of North Texas, Denton, TX (US)

(72) Inventor: Pudur Jagadeeswaran, Denton, TX (US)

(21) Appl. No.: 16/023,753

(22) Filed: Jun. 29, 2018

Related U.S. Application Data
(60) Provisional application No. 62/527,346, filed on Jun. 30, 2017.

(51) Int. Cl. A61K 48/00

(52) U.S. Cl. CPC A61K 48/005; A61P 35/00; A61K 48/0008

(57) ABSTRACT

The present invention includes nucleic acid hybrid molecules capable of entering cells comprising at least one vivo-morpholino oligonucleotide (vivo-MO) comprising a guanidine-rich head conjugated to the 5′ end, and at least one standard oligonucleotide comprising a gene-specific sequence and a standard oligonucleotide pairing sequence, wherein the standard oligonucleotide is bound to the vivo-morpholino oligonucleotide through base pairing to form a hybrid and wherein the vivo-morpholino oligonucleotide pairing sequence is complementary to the standard oligonucleotide pairing sequence.

### CROSS-REFERENCE TO RELATED APPLICATIONS

This application claims priority based on U.S. Provisional Application No. 62/527,346, filed Jun. 30, 2017. The contents of which is incorporated by reference in its entirety.

### TECHNICAL FIELD OF THE INVENTION

The present invention relates in general to the field of gene delivery and more specifically to methods of delivering gRNA and Cas9 RNA nucleotides into cells through a piggy back delivery system.

### STATEMENT OF FEDERALLY FUNDED RESEARCH

None.

### INCORPORATION-BY-REFERENCE OF MATERIALS FILED ON COMPACT DISC

The present application includes a Sequence Listing, which has been submitted in ASCII format via EFS-Web and is hereby incorporated by reference in its entirety. Said ASCII copy, created on Jun. 28, 2018, is named UNTD1031US_SeqList and is 3 kilobytes in size.

### BACKGROUND OF THE INVENTION

Without limiting the scope of the invention, its background is described in connection with biotechnology and genome editing, specifically the intravenous piggy-back delivery of a CRISPR/Cas9 system into organisms for therapeutic purposes.

Genetic Knockdowns are well established in the art to study the function of genes in animal models as well as in cells. Furthermore, delivering the reagents to cells is complex and more often than not, results in poor efficacy and high cell toxicity. Knockdowns have been achieved by the use of antisense oligonucleotides and siRNA, e.g., standard oligonucleotides (SOs) and modified oligonucleotides (MOs and Vivo-MOs as well as phosphorothionates). MOs and Vivo-MOs are more stable than standard oligonucleotides because they are not degraded by nucleases, and thus can be used to inhibit gene functions. The advantage of Vivo-MOs is that they penetrate the cells, and therefore a researcher can deliver them to cells without using special techniques such as microinjection or chemical delivery methods.

Recent advances in genome sequencing techniques and analysis methods have significantly enhanced the ability to identify, catalog, and map genetic factors associated with a diverse range of biological functions and diseases. Precise genome targeting technologies are needed to facilitate systematic reverse engineering of genetic variations, as well as to advance synthetic biological, biotechnological, and medical applications. Editing genomes using the RNA-guided DNA targeting principle of CRISPR-Cas9 (Clustered Regularly Interspaced Short Palindromic Repeats-CRISPR associated proteins) immunity has been recently exploited. The main advantage provided by the bacterial type II based CRISPR-Cas9 system lies in the minimal requirement for programmable DNA interference. Recent studies have demonstrated that the RNA-guided CRISPR-Cas9 system can be employed as an efficient genome editing tool in human cells, mice, zebrafish, drosophila, worms, plants, yeast, and bacteria. The system is versatile, enabling multiplex genome engineering by programming Cas9 to edit several sites in a genome simultaneously by simply using multiple guide RNAs.

U.S. Pat. No. 8,697,359, entitled, “CRISPR-Cas systems and methods for altering expression of gene products” discloses methods, and compositions for altering expression of target gene sequences and related gene products. Provided are vectors and vector systems, some of which encode one or more components of a CRISPR complex, as well as methods for the design and use of such vectors. Also provided are methods of directing CRISPR complex formation in eukaryotic cells and methods for utilizing the CRISPR-Cas system.

U.S. Pat. No. 7,935,816, entitled, “Molecular transporter compositions comprising dendrimeric oligoguanidine with a triazine core that facilitate delivery into cells in vivo,” describes preparations of molecular transporter compositions and their use for transporting bioactive substances into cells in living animals. For in vivo delivery, the composition is covalently linked to the bioactive substance and the resultant composite structure is introduced into the subject. The transporter composition includes multiple guanidine moieties on a dendrimeric scaffold having a triazine core.

U.S. Patent Application Publication No. 2011/0190287, entitled, “Thrombocyte Inhibition Via Vivo-Morpholino Knockdown of Alpha IIB” describes compounds comprising a guanidine-rich head covalently coupled to one or more oligonucleotide antisense sequences which are useful to modulate blood coagulation by affecting the expression of integrin α.IIb or β.3. Also included are pharmaceutical compositions containing these compounds, with or without other therapeutic agents, and well as methods of using these compounds as inhibitors of platelet aggregation, as thrombolytics, and/or for the treatment of other thromboembolic disorders. Vivo-MOs, which include eight guanidine groups dendrimerically arranged in the guanidine-rich head and two synthetic antisense morpholino oligonucleotides, are representative compounds.

### SUMMARY OF THE INVENTION

Morpholino and vivo-morpholino gene knockdown methods were used to study thrombocyte function in zebrafish. Because large-scale knockdown of the entire zebrafish genome using these technologies to study thrombocyte function is prohibitively expensive, the inventors developed an inexpensive gene knockdown method, which uses a hybrid of a control vivo-morpholino and a standard antisense oligonucleotide specific for a gene. This hybrid molecule was shown to piggyback on a control vivo-morpholino, and deliver antisense deoxyoligonucleotides into zebrafish thrombocytes. To validate use of this hybrid molecule in gene knockdowns, the researchers targeted the thrombocyte specific αIIb gene with a hybrid of a control vivo-morpholino and an oligonucleotide antisense to αIIbmRNA. The use of this piggyback technology resulted in degradation of αIIbmRNA and led to thrombocyte functional defect. This known piggyback method to knockdown genes is inexpensive, and one control vivo-morpholino can be used to target many different genes by making many independent gene-specific oligonucleotide hybrids. Thus, this piggyback technology can be utilized for cost-effective large-scale knockdowns of genes to study thrombocyte function in zebrafish.

In one embodiment, the present invention includes a method to treat a patient suspected of having a disease comprising: obtaining a nucleic acid hybrid molecule capable of entering cells comprising at least one oligonucleotide comprising a gene-specific sequence and a oligonucleotide pairing sequence, at least one vivo-morpholino oligonucleotide (vivo-MO) comprising a guanidine-rich head conjugated to the 5′ end, wherein the vivo-morpholino oligonucleotide pairing sequence is complementary to the oligonucleotide pairing sequence, and wherein the oligonucleotide is bound to the vivo-morpholino oligonucleotide through base pairing, forming a hybrid; and contacting the patient with the nucleic acid hybrid molecule. In one aspect, the method further comprises contacting the cell with the nucleic acid hybrid molecule is selected from the group consisting of administering the nucleic acid hybrid molecule to a vertebrate orally, intravenously, intramuscularly, intraperitoneally, subcutaneously, by intranasal instillation, by application to mucous membranes, and by instillation into hollow organ walls or newly vascularized blood vessels. In another aspect, the disease is selected from the group consisting of diabetes, cancer, genetic disorder, diabetes, infectious disease, hemophilia, viral hepatitis, AIDS, genetic disease, thalassemia, sickle cell disease, and Duchene Muscular dystrophy. In one aspect, the method further comprises determining a gene expression of a gene complementary to the gene-specific sequence. In one aspect, the standard oligonucleotide is selected from the group consisting of a DNA oligonucleotide, a RNA, a RNAi, a siRNA, phosphorodithio oligonucleotide, a phosphorothio oligonucleotide, a locked oligonucleotide, and a peptide nucleic acid. In one aspect, the standard oligonucleotide comprises a gene. In one aspect, the guanidine-rich head comprises a dendrimeric octaguinidine.

In another embodiment, the present invention includes a nucleic acid hybrid molecule for treating a patient suspected of having a disease wherein the nucleic acid hybrid molecule comprises: one oligonucleotide comprising a gene-specific sequence and a oligonucleotide pairing sequence, at least one vivo-morpholino oligonucleotide (vivo-MO) comprising a guanidine-rich head conjugated to the 5′ end, wherein the vivo-morpholino oligonucleotide pairing sequence is complementary to the oligonucleotide pairing sequence, and wherein the oligonucleotide is bound to the vivo-morpholino oligonucleotide through base pairing, forming a hybrid. In one aspect, the disease is selected from the group consisting of diabetes, cancer, genetic disorder, diabetes, infectious disease, hemophilia, thalassemia, sickle cell disease, and Duchene Muscular dystrophy. In another aspect, the gene-specific sequence comprises a sequence that is antisense to a mRNA or a pre-mRNA. In another aspect, the gene-specific sequence is complementary to at least one coding DNA, noncoding DNA, or a splice site. In another aspect, the standard oligonucleotide comprises a DNA oligonucleotide. In another aspect, the standard oligonucleotide comprises an RNA. In another aspect, the standard oligonucleotide comprises a siRNA or an RNAi. In another aspect, the standard oligonucleotide comprises a gene. In another aspect, the standard oligonucleotide is selected from the group consisting of phosphorodithio oligonucleotide, phosphorothio oligonucleotide, locked oligonucleotide, and peptide nucleic acid. In another aspect, the standard oligonucleotide pairing sequence is located 3′ of the gene-specific sequence, whereby the standard oligonucleotide has a 5′ overhanging end of 20-30 nucleotides. In another aspect, the morpholino oligonucleotide pairing sequence and the gene-specific oligonucleotide pairing sequence is 12-20 long. In another aspect, the standard, the morpholino, or both oligonucleotides further comprises a guanidine-rich head comprises a dendrimeric octaguinidine.

In another embodiment, the present invention includes a method to treat a patient suspected of having a disease comprising: identifying a subject in need of treatment; designing a nucleic acid hybrid molecule capable of entering cells comprising at least one oligonucleotide comprising a gene-specific sequence and a oligonucleotide pairing sequence, at least one vivo-morpholino oligonucleotide (vivo-MO) comprising a guanidine-rich head conjugated to the 5′ end, wherein the vivo-morpholino oligonucleotide pairing sequence is complementary to the oligonucleotide pairing sequence, and wherein the oligonucleotide is bound to the vivo-morpholino oligonucleotide through base pairing, forming a hybrid; and contacting the patient with the nucleic acid hybrid molecule.

### BRIEF DESCRIPTION OF THE DRAWINGS

For a more complete understanding of the features and advantages of the present invention, reference is now made to the detailed description of the invention along with the accompanying figures.

FIG. 1 is a schematic diagram of the hybrid molecule formation.

FIG. 2 shows gill-bleeding assay.

FIGS. 3a-3b shows blood smear from TG(fli1:EGFP)y1 transgenic fish treated with EGFP-SO/ngVMO hybrid.

FIGS. 4a-4b shows the labeling of zebrafish blood cells due to the uptake of labeled gRNA and Cas9 by piggybacking.

### DETAILED DESCRIPTION OF THE INVENTION

While the making and using of various embodiments of the present invention are discussed in detail below, it should be appreciated that the present invention provides many applicable inventive concepts that can be embodied in a wide variety of specific contexts. The specific embodiments discussed herein are merely illustrative of specific ways to make and use the invention and do not delimit the scope of the invention.

To facilitate the understanding of this invention, a number of terms are defined below. Terms defined herein have meanings as commonly understood by a person of ordinary skill in the areas relevant to the present invention. Terms such as “a”, “an” and “the” are not intended to refer to only a singular entity, but include the general class of which a specific example may be used for illustration. The terminology herein is used to describe specific embodiments of the invention, but their usage does not delimit the invention, except as outlined in the claims.

Genome targeting and editing sequences such as CRISPR are being commonplace in the biotechnological world, and are powerful tools for the treatments of many diseases. CRISPR sequences are transcribed into short RNA sequences capable of guiding the system to matching sequences of DNA. When the target DNA is found, the enzyme Cas9 which is produced by the CRISPR system, binds to the target DNA sequence and cleaves it, shutting the targeted gene off. In other words, CRISPR/Cas9 system does not require the generation of customized proteins to target specific sequences. Using modified versions of Cas9, researchers can activate gene expression instead of cutting the DNA, and these techniques allow researchers to study the gene function. Generally speaking, the method of inserting the CRISPR gene usually involves viral vectors and a high degree of toxicity to the cells. There is a great need for alternative robust systems and techniques for sequence targeting and editing with a wide array of applications. The disclosed invention addresses this need and provides a method for inserting the CRISPR-Cas9 system into an organism without the contraindications of toxicity or the use of viral vectors.

By using an existing intravenous piggyback method and a non-toxic piggyback reagent to deliver the CRISPR-Cas9 system comprising of gRNA and Cas9 RNA into Hematopoietic stem cells or somatic cells, the disclosed technology presents a novel genome targeting and editing technique. The disclosed invention's techniques and analysis methods may significantly simplify the current CRISPR methodology, and accelerate the ability to catalog and map genetic factors associated with a diverse range of biological functions and diseases. The disclosed technology can also treat conditions including, but not limited to, Hemophilia, Sickle Cell disease, and Duchenne Muscular Dystrophy.

Knockdown inhibition is well established genomic tool, and it is known that antisense oligonucleotides can be used to knockdown protein levels by either translational or splice blocking to control cancer and viral infections with the goal of treating human diseases. This knockdown inhibition can be used in model organisms, such as zebrafish, predominantly through the use of Modified Oligonucleotides (MOs), to study functions of proteins in both development and disease, particularly as a gene discovery tool. These MOs are introduced into the yolks of 1-8-cell-stage zebrafish embryos. Due to the cytoplasmic bridges, MOs rapidly diffuse into these cells allowing ubiquitous cytosolic delivery. However, direct cytosolic delivery of MOs into cells has been difficult to achieve with the exception of microinjections. Photoactivatable MOs can be introduced to achieve tissue-specific knockdowns in embryos. And conjugation of dendrimeric octaguinidine to MOs (Vivo-MOs) results in permeability of MOs into cells. Because of this membrane diffusible nature and lack of toxicity, use in human therapy is possible. Vivo-MOs can be employed to evaluate their use in treatment of Duchenne muscular dystrophy.

To utilize the CRISPR-Cas system with the piggyback delivery method using MOs effectively for genome editing without deleterious effects, it is critical to understand the disclosed technology's aspects of engineering and optimization of these genome engineering tools. In one aspect, the invention provides a method for altering or modifying expression of one or more gene products. In another embodiment of the disclosed technology, gRNA and Cas9 RNA are delivered into adult zebrafish cells with a hybrid molecule through the use of gRNA and Cas9 RNA piggyback forward and reverse primers specifically designed to amplify the customized gRNA and Cas9 DNA vector for coagulation factor VIII.

In one example and non-limiting embodiment, a schematic diagram of an embodiment of hybrid molecule can be:

The top strand is the Vivo Morpholino (e.g., 25 nt). * denotes the modification which facilitates the entry of the Vivo-MO. Dots represent base pairing (e.g., 14 bp). The bottom strand standard oligonucleotide. The unpaired region (e.g., 25 nt) of the standard oligonucleotide comprises a gene-specific sequence, e.g., a region that is complimentary to the mRNA of interest, e.g., the target for knockdown.

In one embodiment of the disclosed technology, nucleic acid hybrid molecules made of a non-gene specific Vivo-MO (ngVMO) and a gene specific standard oligonucleotide (SO) are utilized. The hybrid is designed to base pair leaving 5′ overhanging ends. The unbasepaired gene specific SO is, at least partially, antisense to the target mRNA/premRNA. The hybrid molecule has the ability to enter cells because the SO is piggy-backed onto the ngVMO. The present inventors have validated this concept by targeting two proteins, αIIb and EGFP, by using the above piggy-backing strategy and found that expression of both proteins was effectively reduced, resulting in both increased bleeding and loss of EGFP. This approach is less expensive and more efficient compared to a Vivo-MO knockdown. Therefore, these nucleic acid hybrid molecules make large-scale functional genomics a realistic goal rather than a multimillion-dollar undertaking. This approach can be used to inhibit undesired protein expression in a variety of human disorders including AIDS. Furthermore, since RNAs and genes can be piggybacked using ngVMOs, in one embodiment, the present nucleic acid hybrid molecules can be used in gene therapy in cases and to deliver the CRISPR/Cas9 system to a specific target when protein expression is desirable.

Zebrafish SO/ngVMO injections to generate knockdowns: A ngVMO 5′-CCTCTTACCTCAGTTACAATTTATA-3′ (SEQ ID NO: 1) was purchased from Gene-Tools LLC, Philomath Oreg. A SO was designed so that it can hybridize both to ngVMO (14 bp) and to αIIb pre-mRNA at the donor splice site of exon 20 (25 bp), 5′-GGAAGTGACTAAACCCTCACCTCATTATAAATTGTAACTG-3′ (SEQ ID NO: 2). A control SO that can hybridize to ngVMO and has a complementary sequence corresponding to the antisense sequence portion of the above SO, 5′-ATGAGGTGAGGGTTTAGTCACTTCCTATAAATTGTAACTG-3′ (SEQ ID NO: 3) was designed. Two other SOs were designed: one that targets EGFP mRNA and the other its control, 5′-TGTACATAACCTTCGGGCATGGCACTATAAATTGTAACTG-3′ (SEQ ID NO: 4) and 5′-GTGCCATGCCCGAAGGTTATGTACATATAAATTGTAACTG-3′ (SEQ ID NO: 5), respectively. All SOs and their controls were purchased from Invitrogen, Carlsbad, Calif. 4.5 μl of 0.5 mM ngVMO was mixed with 4.5 μl of 0.5 mM SO and 1 μl 10× phosphate buffered saline, pH 7.4 (PBS). The mixture was heated at 90 degree C. and slowly cooled to room temperature so that the SO and ngVMO could hybridize. 5 μl of this hybridized SO/ngVMO was used to inject an adult zebrafish intravenously. αIIb-SO/ngVMO hybrid was injected into wild type zebrafish whereas EGFP-SO/ngVMO hybrid was injected into TG(fli1:EGFP)y1 zebrafish, which carries the transgene of the FLI1 gene promoter driving GFP and in which all thrombocytes are GFP positive.

RT-PCR: Zebrafish blood was centrifuged at 500 g and the white cell layer was used in the cell to cDNA kit (Agilent Technologies, LaJolla, Calif.) to amplify the αIIb mRNA. The present inventors designed forward 5′-AGTGCTGCATGGACAAAGTG-3′ (SEQ ID NO: 6) and reverse 5′-GGTTCTCCACCTGTTCCAGA-3′ (SEQ ID NO: 7) primers for exons 18 and 22, respectively; these were synthesized by Biosynthesis, Lewisville, Tex. These primers were used to amplify the 396 bp product. In the case of exon skipping, the predicted product is 149 base pair. These RT-PCR products were resolved on 1.5% agarose gels.

In one embodiment, the disclosed technology utilizes Vivo-MO technology to inhibit thrombocyte function in adult zebrafish for the first time by injecting thrombocyte-specific αIIb Vivo-MOs intravenously into adult zebrafish; thereby, establishing a proof of principle and providing a basis to target two other novel candidate thrombocyte receptors to knockdown thrombocyte function, and to evaluate the function of novel genes involved in, e.g., hemostatic pathways.

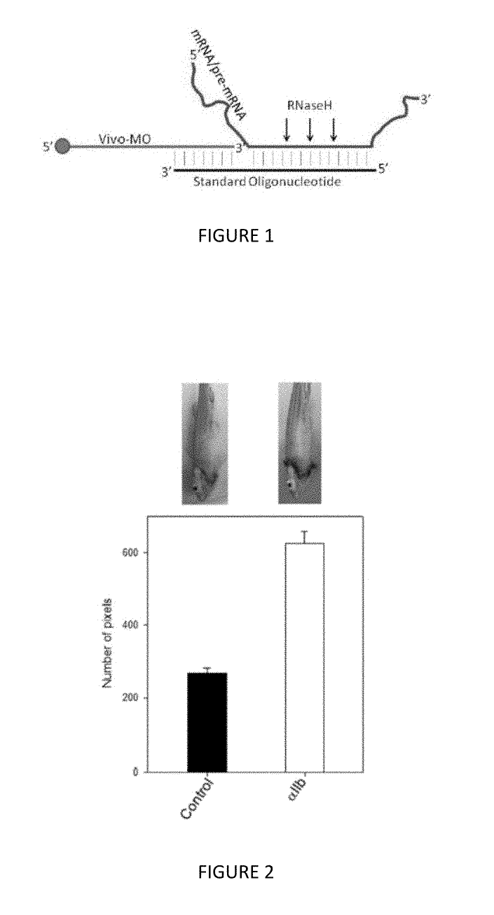

FIG. 1 is a schematic diagram of the hybrid molecule formation that illustrates the mechanism behind piggybacking. The possible hybrid molecule formation (base pairing indicated by small vertical bars) between non-gene Vivo-Morpholino, ngVMO (shown in green) and standard oligonucleotide, SO (shown in blue) as well as hybrid formation between SO and either mRNA or pre-mRNA (shown in red). Arrows show RNaseH cleavage of the mRNA/pre-mRNA portion of the RNA-DNA hybrid. Closed circle represents dendrimeric octaguinidine conjugated at the 5′ end of ngVMO. If an SO is synthesized such that it is complementary to the target mRNA/pre-mRNA on the 5′ side and complementary to a non-gene specific Vivo-MO (ngVMO) on the 3′ side, then this SO/ngVMO hybrid will enter the cell since the SO is ‘piggy-backed’ onto the ngVMO. Once inside the cell, in one embodiment, the SO portion binds to the target mRNA/pre-mRNA and leads to the cleavage of the target mRNA by the endogenous RNaseH mechanism. In other embodiments, the SO participates in splice and/or translation blocking using a mechanism similar to that of Vivo-MO targeting. In yet another embodiment, the SO allows for the transport of the CRISPR/Cas9 system into a cell.

FIG. 2 shows gill-bleeding assay. Zebrafish were photographed and the number of red pixels representing the bleeding were measured in control SO hybrid treated (Control) and αIIb-SO/ngVMO hybrid (αIIb) treated zebrafish (N=6). p value is <0.001 between the Control and αIIb. FIG. 2 illustrates thrombocyte functionality with the use of a gill bleeding assay. Gill bleeding was induced by placing the zebrafish in a petri dish containing 50 ml of 50 μM NaOH. The zebrafish were anesthetized in 50 ml of 2 mM tricaine (Sigma-Aldrich, St. Louis, Mo.) for 3 minutes prior to placement in NaOH. The zebrafish were photographed with a NIKON™ E995 Coolpix camera, and the red pixels were counted by ADOBE PHOTOSHOP™ software 7.0 and used to quantify bleeding. This gill-bleeding assay was performed to show that the reduction in mRNA producing αIIb resulted in greater bleeding. As FIG. 2 summarizes, the zebrafish treated with αIIb-SO/ngVMO hybrid exhibited more bleeding compared to the fish treated with control hybrid.

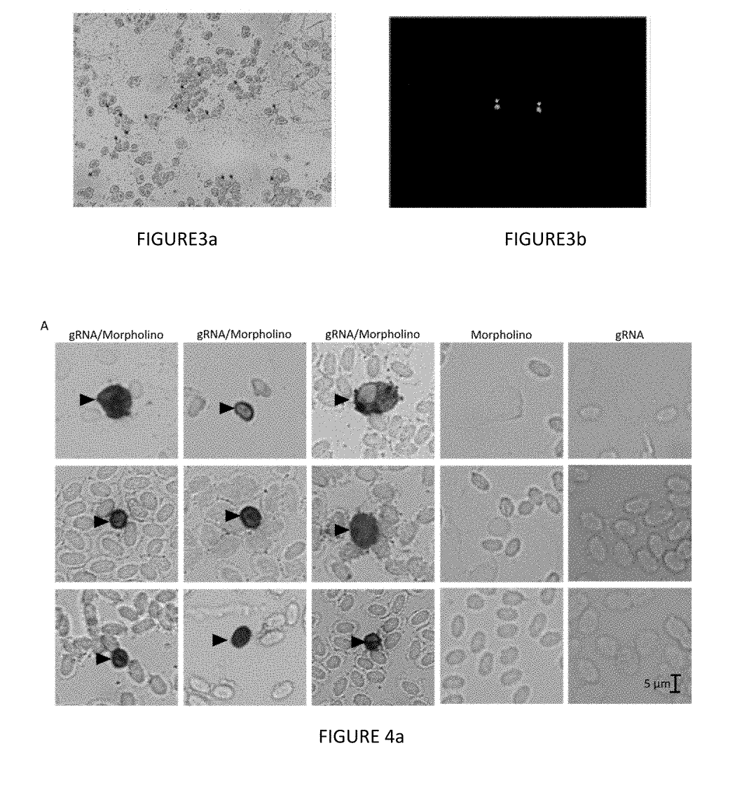

FIGS. 3a and 3b are the brightfield and fluorescence images respectively. Note that there are several thrombocytes lacking GFP fluorescence shown by black arrows in FIG. 3a. Only a few thrombocytes are GFP positive shown by red arrows in FIG. 3a. White arrows in FIG. 3b show the GFP thrombocytes. EGFP synthesis was inhibited in thrombocytes that are GFP+ by injecting EGFP-SO/ngVMO hybrid intravenously into TG(fli1:EGFP)y1 transgenic zebrafish. Twenty-four hours after injection, a blood smear was prepared from these transgenic fish and GFP fluorescence in thrombocytes was observed: Almost 70-80% of thrombocytes lost GFP fluorescence. The present inventors demonstrate that, as a non-limiting example, direct injection of αIIb-SO/ngVMO hybrid intravenously into zebrafish inhibits αIIb and thus reduces thrombocyte aggregation. The RT-PCR results show that alternatively spliced αIIb mRNA is generated resulting in a 149 bp product (confirmed by sequence analysis) which provides evidence that the αIIb-SO/ngVMO hybrid is effectively penetrating thrombocytes. Interestingly, the effect of αIIb-SO/ngVMO hybrid on thrombocyte aggregation was also observed in 24 hrs. This result is similar to what the inventors observed with αIIb Vivo-MO [9]. This documents that, in addition to the alternative splicing due to splice blocking, the pre-mRNA is cleaved by RNaseH. Therefore, the EGFP study was conducted: SO were used that targets the EGFP mRNA sequence located approximately in the middle of translational initiation and terminator codons. Thus, the target is neither a translational blocker nor a splice blocker. Thus, reduction in EGFP in thrombocyte is due to the degradation of EGFP RNA by RNaseH mechanism. The results demonstrated 70-80% GFP-thrombocytes showing that the EGFP-SO/ngVMO hybrid method is very efficient and degrades EGFP RNA by RNaseH mechanism.

In another embodiment of the disclosed technology, gRNA and Cas9 RNA are delivered into adult zebrafish cells with the use of gRNA piggyback forward and reverse primers were designed to amplify the customized gRNA vector DNA for coagulation factor VIII. The gRNA piggyback forward primer comprises the first 18 nucleotides of the T7 promoter sequence followed by 12 nucleotides of the 5′ end of factor VIII sequence. The gRNA piggyback reverse primer had at its 5′ end, 15 nucleotides that are complementary to 3′ end of control vivo morpholino (5′-TATAAATTGTAACTG-3′) (SEQ ID NO: 8) followed by the sequence complementary to the 3′ end of the gRNA sequence. Cas9 piggyback forward and reverse primers were designed to amplify Cas9 plasmid DNA. Cas9 piggyback forward primer comprised 18 nucleotides of the SP6 promoter sequence followed by 12 nucleotides of 5′ end of the Cas9 vector sequence. Cas9 piggyback reverse primer had at its 5′ end 15 nucleotides that are complementary to 3′ end of control vivo morpholino as described above followed by the sequence complementary to the 3′ end of the Cas9 sequence representing the SV40 terminator site sequence. Both the gRNA and Cas9 plasmids were amplified with the primers sequences shown below:

gRNA piggyback forward
SEQ ID NO: 9
GTAATACGACTGAGTATAGGGACATTTCTC
gRNA piggyback reverse
SEQ ID NO: 10
CAGTTACAATTTATAGATCCGCACCGACTC
Cas9 piggyback forward
SEQ ID NO: 11
ATTTAGGTGACACTATAGAATACAAGCTAC
Cas9 piggyback reverse
SEQ ID NO: 12
CAGTTACAATTTATAGTTTATTGCAGCTTA

The amplified gRNA and Cas9 DNA was transcribed with T7 RNA polymerase and SP6 polymerase, respectively. Then transcribed again with digoxigenin (DIG) labeled UTP. The RNA and control vivo morpholino were incubated at 94° C. for 5 minutes and cooled to 4° C. to form a complex. The labeled RNA/morpholino, labeled RNA alone (control), and morpholino alone (control) were injected separately into adult fish intravenously and incubated for 30 minutes. Blood was then collected, and a blood smear prepared on a microscope slide. The slides were incubated in 4% paraformaldehyde for 15 minutes to affix the cells. The slides were then washed three times with 1×PBS. Subsequently, the slides were incubated in 0.5% saponin for 1.5 hours to permeabilize the cells. The slides were rinsed three times with 1×PBS. A blocking buffer (10% NGS, 2% BSA, 0.1% saponin) was prepared and incubated with the slides overnight at 4° C. The slides were then incubated with anti-DIG antibody in the blocking buffer, at a ratio of 1:500, for 1.5 hours. The slides were rinsed three times with 1×PBS. NBT/BCIP staining solution was added to the slides until color developed (about 1.5 hours). The slides were viewed under a microscope at 20× magnification, and pictures were taken.

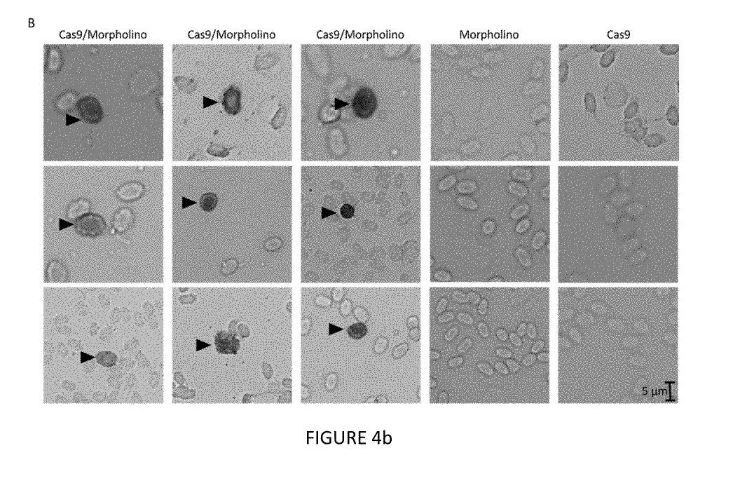

FIGS. 4a-4b shows the labeling of zebrafish blood cells due to the uptake of labeled gRNA and Cas9 by piggybacking. FIGS. 4a and 4b illustrate that the blood smear showed approximately 20% of 100 cells were observed to be blue in color in gRNA/control vivo morpholino injected blood cells. FIG. 4a shows that fish were injected with either gRNA/control vivo morpholino complex, morpholino, or gRNA. After injection, the blood smears were probed with anti-DIG antibodies, and photographs were taken at 20× magnification. The black arrows show a variety of cell types that took up the gRNA/control vivo morpholino complex. FIG. 4b shows zebrafish injected with either Cas9/control vivo morpholino complex, morpholino, or Cas9 RNA. After injection, the blood cells were probed with anti-DIG antibodies, and pictures were taken at 20× magnification. The black arrows show a variety of cell types that took up the Cas9/control vivo morpholino complex. However, only 15% of 100 cells that we observed were blue in color in Cas9 RNA/control vivo morpholino injected blood cells. Thus, we found that both gRNA and Cas9 RNA were taken up by the cells in samples that had both RNAs (either gRNA or Cas9 RNA) and control vivo morpholinos. The control vivo morpholino alone and the RNA alone did not show any blue color. Interestingly, it should be noted that a greater number of thrombocytes (approximately ¾ of blue colored cells) showed blue color as compared to the other types (approximately ¼ of blue colored) of blood cells. Overall, this study validates the point that the CRISPR gRNA and Cas9 were successfully taken up by the vivo morpholino cells as a result of the piggybacking.

Herein, the disclosed technology comprises many embodiments with tremendous applicability, not only to identify functions of novel genes in thrombocytes but also in other accessible blood cells as well as in highly vascular organs. Since it is possible to deliver SO and CRISPR/Cas9 hybrid molecules to any hematopoietic or somatic cells, it is also possible to use these reagents not only as an antithrombotic agent, but also as an agent to correct other hematological and muscular disorders. The efficiency of knockdowns can be increased by designing multiple SOs for the same target RNA. In light of this, the mechanisms of these and many other disorders can be revealed. In addition, RNAs and genes like the CRISPR/CAs9 can be delivered easily into cells by complexing with a ngVMO, thus, this technology is applicable to gene therapy as well as RNA/protein therapies. Furthermore, efficient delivery of siRNA piggy-backed on ngVMO into the cells in whole organism should also be feasible. This allows for an additional, complementary approach to inhibit protein expression. Thus, the present technology has numerous implications for genome identification, targeting, and drug delivery.

It is contemplated that any embodiment discussed in this specification can be implemented with respect to any method, kit, reagent, or composition of the invention, and vice versa. Furthermore, compositions of the invention can be used to achieve methods of the invention.

It will be understood that particular embodiments described herein are shown by way of illustration and not as limitations of the invention. The principal features of this invention can be employed in various embodiments without departing from the scope of the invention. Those skilled in the art will recognize, or be able to ascertain using no more than routine experimentation, numerous equivalents to the specific procedures described herein. Such equivalents are considered to be within the scope of this invention and are covered by the claims.

All publications and patent applications mentioned in the specification are indicative of the level of skill of those skilled in the art to which this invention pertains. All publications and patent applications are herein incorporated by reference to the same extent as if each individual publication or patent application was specifically and individually indicated to be incorporated by reference.

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or the alternatives are mutually exclusive, although the disclosure supports a definition that refers to only alternatives and “and/or.” Throughout this application, the term “about” is used to indicate that a value includes the inherent variation of error for the device, the method being employed to determine the value, or the variation that exists among the study subjects.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, AB, BBC, AAABCCCC, CBBAAA, CABABB, and so forth. The skilled artisan will understand that typically there is no limit on the number of items or terms in any combination, unless otherwise apparent from the context.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. All such similar substitutes and modifications apparent to those skilled in the art are deemed to be within the spirit, scope and concept of the invention as defined by the appended claims.

### REFERENCES

[1] R. M. Hudziak, J. Summerton, D. D. Weller, and P. L. Iversen, Antiproliferative effects of steric blocking phosphorodiamidate morpholino antisense agents directed against c-myc. Antisense Nucleic Acid Drug Dev 10 (2000) 163-76.

[2] C. S. Cobbs, T. R. Whisenhunt, D. R. Wesemann, L. E. Harkins, E. G. Van Meir, and M. Samanta, Inactivation of wild-type p53 protein function by reactive oxygen and nitrogen species in malignant glioma cells. Cancer Res 63 (2003) 8670-3.

[3] A. Nasevicius, and S. C. Ekker, Effective targeted gene ‘knockdown’ in zebrafish. Nat Genet 26 (2000) 216-20.

[4] M. Gregory, R. Hanumanthaiah, and P. Jagadeeswaran, Genetic analysis of hemostasis and thrombosis using vascular occlusion. Blood Cells Mol Dis 29 (2002) 286-95.

[5] I. A. Shestopalov, S. Sinha, and J. K. Chen, Light-controlled gene silencing in zebrafish embryos. Nat Chem Biol 3 (2007) 650-1.

[6] P. A. Morcos, Y. Li, and S. Jiang, Vivo-Morpholinos: a non-peptide transporter delivers Morpholinos into a wide array of mouse tissues. Biotechniques 45 (2008) 613-4, 616, 618 passim.

[7] J. D. Moulton, and S. Jiang, Gene knockdowns in adult animals: PPMOs and vivo-morpholinos. Molecules 14 (2009) 1304-23.

[8] B. Wu, Y. Li, P. A. Morcos, T. J. Doran, P. Lu, and Q. L. Lu, Octa-guanidine morpholino restores dystrophin expression in cardiac and skeletal muscles and ameliorates pathology in dystrophic mdx mice. Mol Ther 17 (2009) 864-71.

[9] S. Kim, U. P. Radhakrishnan, S. K. Rajpurohit, V. Kulkarni, and P. Jagadeeswaran, Vivo-Morpholino knockdown of alphaIIb: A novel approach to inhibit thrombocyte function in adult zebrafish. Blood Cells Mol Dis 44 169-74.

What is claimed is:

1. A method to treat a patient suspected of having a disease comprising:
obtaining a nucleic acid hybrid molecule capable of entering cells comprising at least one oligonucleotide comprising a gene-specific sequence and a oligonucleotide pairing sequence, at least one vivo-morpholino oligonucleotide (vivo-MO) comprising a guanidine-rich head conjugated to the 5′ end, wherein the vivo-morpholino oligonucleotide pairing sequence is complementary to the oligonucleotide pairing sequence, and wherein the oligonucleotide is bound to the vivo-morpholino oligonucleotide through base pairing, forming a hybrid; and
contacting the patient with the nucleic acid hybrid molecule.

2. The method of claim 1, wherein contacting the cell with the nucleic acid hybrid molecule is selected from the group consisting of administering the nucleic acid hybrid molecule to a vertebrate orally, intravenously, intramuscularly, intraperitoneally, subcutaneously, by intranasal instillation, by application to mucous membranes, and by instillation into hollow organ walls or newly vascularized blood vessels.

3. The method of claim 1, wherein the disease is selected from the group consisting of diabetes, cancer, genetic disorder, diabetes, infectious disease, hemophilia, viral hepatitis, AIDS, genetic disease, thalassemia, sickle cell disease, and Duchene Muscular dystrophy.

4. The method of claim 1, further comprising determining a gene expression of a gene complementary to the gene-specific sequence.

5. The method of claim 1, wherein the standard oligonucleotide is selected from the group consisting of a DNA oligonucleotide, a RNA, a RNAi, a siRNA, phosphorodithio oligonucleotide, a phosphorothio oligonucleotide, a locked oligonucleotide, and a peptide nucleic acid.

6. The method of claim 1, wherein the standard oligonucleotide comprises a gene.

7. The method of claim 1, wherein the guanidine-rich head comprises a dendrimeric octaguinidine.

8. A nucleic acid hybrid molecule for treating a patient suspected of having a disease wherein the nucleic acid hybrid molecule comprises:
one oligonucleotide comprising a gene-specific sequence and a oligonucleotide pairing sequence, at least one vivo-morpholino oligonucleotide (vivo-MO) comprising a guanidine-rich head conjugated to the 5′ end, wherein the vivo-morpholino oligonucleotide pairing sequence is complementary to the oligonucleotide pairing sequence, and wherein the oligonucleotide is bound to the vivo-morpholino oligonucleotide through base pairing, forming a hybrid.

9. The nucleic acid hybrid of claim 8, wherein the disease is selected from the group consisting of diabetes, cancer, genetic disorder, diabetes, infectious disease, hemophilia, thalassemia, sickle cell disease, and Duchene Muscular dystrophy.

10. The nucleic acid hybrid of claim 8, wherein the gene-specific sequence comprises a sequence that is antisense to a mRNA or a pre-mRNA.

11. The nucleic acid hybrid of claim 8, wherein the gene-specific sequence is complementary to at least one coding DNA, noncoding DNA, or a splice site.

12. The nucleic acid hybrid of claim 8, wherein the standard oligonucleotide comprises a DNA oligonucleotide.

13. The nucleic acid hybrid of claim 8, wherein the standard oligonucleotide comprises a RNA.

14. The nucleic acid hybrid of claim 8, wherein the standard oligonucleotide comprises a siRNA or a RNAi.

15. The nucleic acid hybrid of claim 8, wherein the standard oligonucleotide comprises a gene.

16. The nucleic acid hybrid of claim 8, wherein the standard oligonucleotide is selected from the group consisting of phosphorodithio oligonucleotide, phosphorothio oligonucleotide, locked oligonucleotide, and peptide nucleic acid.

17. The nucleic acid hybrid of claim 8, wherein the standard oligonucleotide pairing sequence is located 3′ of the gene-specific sequence, whereby the standard oligonucleotide has a 5′ overhanging end of 20-30 nucleotides.

18. The nucleic acid hybrid of claim 8, wherein the morpholino oligonucleotide pairing sequence and the gene-specific oligonucleotide pairing sequence is 12-20 long.

19. The nucleic acid hybrid of claim 8, wherein the standard, the morpholino, or both oligonucleotides further comprises a guanidine-rich head comprises a dendrimeric octaguinidine.

20. A method to treat a patient suspected of having a disease comprising:
identifying a subject in need of treatment;
designing a nucleic acid hybrid molecule capable of entering cells comprising at least one oligonucleotide comprising a gene-specific sequence and a oligonucleotide pairing sequence, at least one vivo-morpholino oligonucleotide (vivo-MO) comprising a guanidine-rich head conjugated to the 5′ end, wherein the vivo-morpholino oligonucleotide pairing sequence is complementary to the oligonucleotide pairing sequence, and wherein the oligonucleotide is bound to the vivo-morpholino oligonucleotide through base pairing, forming a hybrid; and
contacting the patient with the nucleic acid hybrid molecule.
